(19) United States
(12) Patent Application Publication
(10) Pub. No.: US 2012/0253137 A1
(43) Pub. Date: Oct. 4, 2012

(54) SURGICAL ILLUMINATION SYSTEM

(75) Inventors: Alex Vayser, Mission Viejo, CA (US); Kenneth Trauner, San Francisco, CA (US)

(73) Assignee: Invuity, Inc., San Francisco, CA (US)

(21) Appl. No.: 13/245,638

(22) Filed: Sep. 26, 2011

Related U.S. Application Data
(63) Continuation of application No. 11/356,758, filed on Feb. 17, 2006, now abandoned.
(60) Provisional application No. 60/654,357, filed on Feb. 18, 2005.

(51) Int. Cl. A61B 1/06

(52) U.S. Cl. 600/249

(57) ABSTRACT

A surgical illumination system, according to the present disclosure may include one or more illumination elements mounted on or in a collar body or surgical retractor pin that may also include control electronics. The surgical illumination system affords controllable direct illumination of a deep cavity surgical site and may be moved independent of any surgeon or surgical instrument. The collar body or pin provides heat conductivity to remove heat generated by the illumination elements. The collar body is adapted for engagement of Steinman style surgical retractor pins and to afford optimal heat transfer to the retractor pin. Energy for the illumination elements may be provided by batteries or other suitable source through a cable or wireless link.

### CROSS-REFERENCE

The present application is continuation of U.S. patent application Ser. No. 11/356,758 (Attorney Docket No. 40556-710.201) filed Feb. 17, 2006, which is a non-provisional of U.S. Provisional Patent Application No. 60/654,357 (Attorney Docket No. 40556-710.101) filed Feb. 18, 2005; the entire contents of which are incorporated herein by reference.

### FIELD OF THE INVENTION

The inventions described below relate the field of surgical instruments, and more specifically to illumination pins for use in surgical cavities.

### BACKGROUND OF THE INVENTION

Existing technology for illumination during surgical/medical procedures is currently limited to over-head illumination. This illumination comes from either overhead lighting or head mounted fiber optic systems. Traditional overhead lighting systems face numerous limitations. Direct exposure of the surgical field from the overhead source is required. Changes in patient or surgeon positioning may interfere with the field illumination. Frequent adjustments of the light source represents an inconvenience for the surgeon and disrupts the surgical flow. Overhead lighting is frequently inadequate for surgery in deeper cavities where more intense focused illumination may be required. In addition, where multiple surgeons are participating, the alignment of the surgeons head frequently interferes with the remote illumination and prevents light from reaching the surgical field.

Head mounted fiber optic systems are used frequently for more limited surgical exposures. However, head mounted fiber optic systems have limitations. For example, the surgeon is tethered by the light cord attached to the headset, limiting mobility in the operating room. Fiber optic devices are often associated with head and neck fatigue from frequent or prolonged use. Head mounted fiber optic systems may also require the surgeon to maintain a steady head neck position to provide a constant and steady illumination of the field. Also, the use of remote light sources and fiber bundles introduces tremendous inefficiencies into the system. An approximate 10% loss per foot of cable a 300 Watt light source and a 10 ft cable will only provide just a few watts of illumination. The introduction and popularity of minimally invasive surgical techniques, has raised the demand for the delivery of high intensity light through minimal surgical incisions into deep cavity surgical fields.

Some light delivery devices have been developed for delivery of light from a remote, high intensity light sources to the surgical field. Conventional devices consist of bundles of optical fibers directly attached to surgical retractors to illuminate the surgical field. These light delivery devices are connected via fiber optic cable to a high intensity light source. While these devices provide a technique for directly illuminating the surgical field, they are cumbersome. Having these conventional devices directly tethered to the retractors, they provide limited illumination. The fiber bundles are inconvenient, interfere with access, destabilize the retractor positioning, and they provide inefficient illumination.

What is needed is a source of surgical illumination for deep cavity surgery that may be directed independent of the surgeon and the surgical instruments.

### SUMMARY OF THE INVENTION

A surgical illumination system, according to the present disclosure may include one or more illumination elements mounted on or in a collar body or surgical retractor pin that may also include control electronics. The surgical illumination system affords controllable direct illumination of a deep cavity surgical site and may be moved independent of any surgeon or surgical instrument. The collar body or pin provides heat conductivity to remove heat generated by the illumination elements. The collar body is adapted for engagement of Steinman style surgical retractor pins and to afford optimal heat transfer to the retractor pin. Energy for the illumination elements may be provided by batteries or other suitable source through a cable or wireless link.

Recently, advances in solid state lights such as light emitting diode (LED) technology have generated the potential to provide high intensity illumination from a small device. A surgical illumination collar, pin, or pin and collar combination according to the present disclosure may include one or more light sources such as an LED, mounting collar, control electronics, and an energy source.

An illumination collar may be compatible with any standard surgical retractor pins such as Steinman Pins. One or more illumination collars may each be placed on conventional retractor pins to provide direct illumination to a deep cavity surgical site. The illumination elements may have any suitable orientation on the collar, and a collar may include illumination elements have different orientations, or arrays of illumination elements to provide selectable illumination. Illumination provided by a device independent of the surgeon and the surgical tools permits the illumination to be directed as needed with minimal interference and without limiting access to the surgical site.

In another aspect of the present disclosure, one or more illumination elements may be included in a surgical retractor pin such as a Steinman pin along with an energy source, control electronics and connections between the energy source and the illumination elements. In this configuration, the pin may include one or more areas of low heat conductive material to control the flow of heat produced by the illumination elements.

A surgical illumination system, according to the present disclosure may include one or more illumination elements mounted on a collar body or surgical retractor pin that may also include control electronics. The surgical illumination system affords controllable direct illumination of a deep cavity surgical site and may be moved independent of any surgeon or surgical instrument. The collar body or pin provides heat conductivity to remove heat generated by the illumination elements. The collar body is adapted for engagement of Steinman style surgical retractor pins and to afford optimal heat transfer to the retractor pin. Energy for the illumination elements may be provided by batteries through a cable or wireless link.

In another aspect of the present disclosure illumination elements may be any suitable incandescent or solid state devices such as LEDs. Illumination elements may adopt any suitable orientation relative to each other and may have any suitable configuration and or color, such as a white surface mount LED. Other colors or combinations of colors for illumination elements may permit selectable frequency of illumination for a surgical site to enable illumination or therapy. For example, an array of illumination elements may include a red LED, a green LED, and a blue LED or any other suitable combination. The array elements may be collocated in any suitable arrangement to enable any combination of the microchips or other illumination element to be illuminated by control electronics for illumination or therapeutic benefit.

Illumination elements may also be configured to emit polarized light using any suitable technique such as polarizing film.

A surgical illumination system according to the present disclosure may include one or more retractor pins, one or more illumination collars frictionally engaging one or more of the one or more retractor pins, one or more light elements secured to each of the one or more illumination collars, a power source, an energy path connecting the power source to the one or more light elements elements, and a control element controlling the application of power to each of the one or more light elements.

An alternate surgical illumination system may include one or more illuminating retractor pins, each illuminating retractor pin including one or more light elements, a power source providing power to each or the one or more light elements, and a control element controlling the application of power to each of the one or more light elements.

Another surgical illumination system according to the present disclosure may include one or more retractor pins, one or more illumination collars frictionally engaging one or more of the one or more retractor pins, one or more light elements secured to each of the one or more illumination collars, a power source providing power to each or the one or more light elements, a control element controlling the application of power to each of the one or more light elements and one or more illuminating retractor pins, each illuminating retractor pin including one or more light elements, a power source providing power to each or the one or more light elements, and a control element controlling the application of power to each of the one or more light elements.

### BRIEF DESCRIPTION OF THE DRAWINGS

FIG. 1 a perspective view of a surgical site illuminated using a surgical illumination system according to the present disclosure.

FIG. 2 is a side view of a surgical illumination collar according to the present disclosure.

FIG. 3 is a side view of the surgical illumination retractor pin according to the present disclosure.

FIG. 4 is a side view of a surgical illumination retractor pin with alternate illumination element arrangements according to the present disclosure.

### DETAILED DESCRIPTION OF THE INVENTION

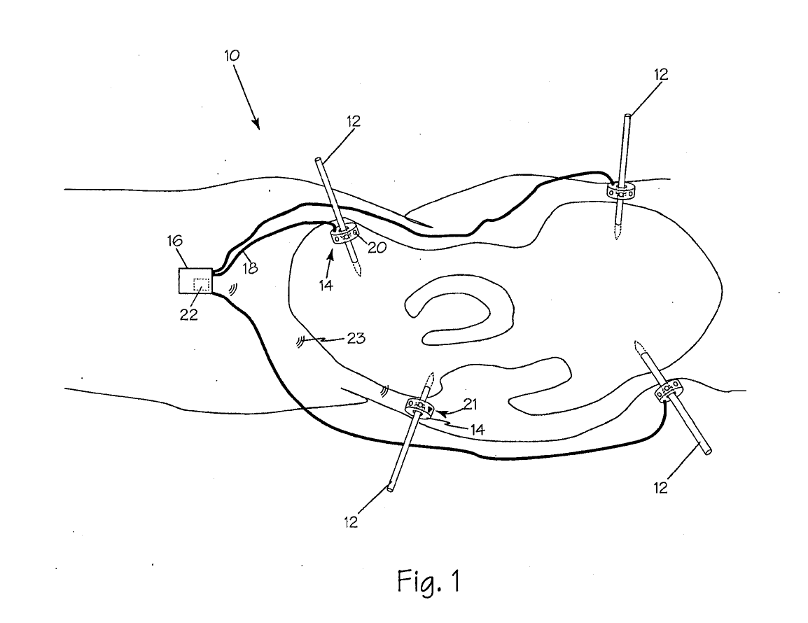

Surgical illumination system 10 of FIG. 1 may include one or more light elements or light element arrays such as LED 20 and array 21 mounted in an illumination collar such as collars 14. Power source 16 may provide energy, under control of control electronics 22, to one or more collars 14 through any suitable cable 18 or through a wireless link such as link 23. Power source 16 may be one or more batteries or a power supply connected to a source of AC power.

In use, surgical illumination collar 14 may be engaged on any suitable surgical retractor such as Steinman pin 12 to provide illumination to a surgical site. Collar 14 may be rotated on Steinman pin 12 to provide illumination as needed and afford the surgeon an optimal view of the surgical site and adjacent tissue and Collar 14 may frictionally engage the surgical retractor to retain a selected orientation.

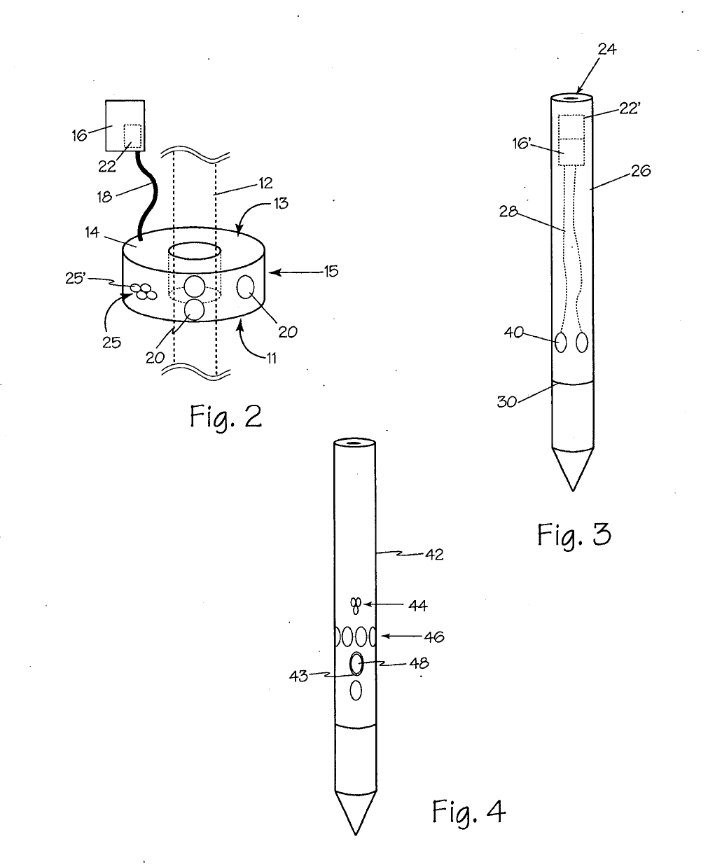

Referring now to FIG. 2, collar 14 provides a mounting surface for one or more light elements such as LEDs 20 and or light array 25. Collar 14 may also operate as a heat sink to conduct heat generated by the light elements from the surgical site. Engagement between collar 14 and Steinman pin 12 may permit the relative orientation between the collar and the pin to be retained as set by a user, and may also permit heat to be transferred from collar 14.

Light elements such as LEDs 20, array 25 or other may be secured or incorporated into the collar at one or more locations about the circumference of side 15, or on surface 11 and or surface 13. Light elements may have any suitable configuration and or color, such as a white surface mount LED.

An array of light elements such as array 25 may include one or more light elements 25′ with any suitable light characteristics such as color, frequency, or polarization or any other suitable combination.

Alternatively, light elements 20 may be organic or polymer LEDs, OLED, PLED respectively, that may have another diffuser filit applied on top of the OLED film to direct the light in a desired direction and/or shape the light in a desired shape, such as circular, square, rectangle, triangle, etc. The light elements may be arrayed to include individual elements having different characteristics such as color, polarization, intensity, focus, direction or any other suitable characteristic. For example, an array of illumination elements such as array 25 of FIG. 2, or arrays 42 or 44 of FIG. 4 may include individual elements having different characteristics such as a red LED, a green LED, and a blue LED or any other suitable combination. The array elements may be collocated in any suitable arrangement to enable any individual element or any suitable combination of the elements to be illuminated by control electronics for illumination and or therapeutic benefit.

Control electronics 22 may be contained within collar 14 or included with power source 16 in a common housing.

Surgical illumination system 10 may also include one or more illumination retractor pins such as retractor pin 26 of FIG. 3. Retractor pin 26 may include internal energy source 16′, internal control electronics 22′ and one or more light elements such as LED 40 electrically connected to energy source 16′ and control electronics 22′ through internal cables such as cable 28. Control electronics may be controlled using external actuators such as actuator 24, or using wireless signals.

Alternatively, a self contained illumination retractor pin such as retractor pin 26 may be controlled by control electronics 22 when pin 26 is part of an array such as in FIG. 1.

Light elements such as LED's 40 may have any suitable configuration and or color, such as a white surface mount LED. To prevent undesired heat transfer from any light elements such as LEDs 40, retractor pin 26 may include heat shield or other suitable heat resistive layer or layers such as layer 30 composed of material with low thermal conductivity.

Referring now to FIG. 4, surgical retractor 42 may include one or more light elements in any suitable arrangement or combination such as array 44 and or array 46.

A surgical illumination system according to the 15 present disclosure may also include any suitable layers, lenses, films or other to modify or control the light from any light elements such as light 48. For example, film 43 may be overlaid on one or more light elements 48 to provide polarized light. Any suitable combination of films, layers and lenses may be used.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical illumination system comprising:
an elongate surgical pin having a first end and a second end;
an illumination collar having a light element mounting surface and frictionally engaging the surgical pin anywhere between the first end and the second end;
one or more light elements mounted in the mounting surface of the illumination collar;
a power source;
an energy path connecting the power source to the one or more light elements; and
a control element controlling the application of power to each of the one or more light elements,
wherein illumination collar position is rotatably adjustable around the surgical pin, and
wherein the surgical pin acts as a heat sink for optimal removal of heat generated by the one or more light elements.

2. The surgical illumination system of claim 1, wherein the power source is one or more batteries.

3. The surgical illumination system of claim 1, wherein the power source is derived from AC power.

4. The surgical illumination system of claim 1, wherein the energy path is one or more wires.

5. The surgical illumination system of claim 1, wherein the energy path is wireless.

6. The surgical illumination system of claim 1, wherein the surgical pin includes a heat resistive layer.

7. The surgical illumination system of claim 1, wherein the one or more light elements are light emitting diodes.

8. The surgical illumination system of claim 1, wherein the one or more light elements are organic light emitting diodes.

9. The surgical illumination system of claim 1, wherein the one or more light elements are polymer light emitting diodes.

10. The surgical illumination system of claim 1, wherein the one or more light elements includes at least one array of light elements wherein each light element of the array may be separately energized and each light element of the array produces light of a different frequency.

11. The surgical illumination system of claim 1, wherein at least one of the one or more light elements includes a polarizing film.

12. The surgical illumination system of claim 1, wherein at least some of the light elements are mounted in a surface which circumscribes the collar.

13. The surgical illumination system of claim 1, wherein at least some of the light elements are mounted in a front surface of the collar.

14. A surgical illumination system comprising:
one or more sterile illumination retractor pins, each sterile illumination retractor pin having one or more light elements incorporated within the sterile illumination retractor pin;
a power source providing power to each or the one or more light elements; and
a control element controlling the application of power to each of the one or more light elements,
wherein the retractor pin acts as a heat sink for optimal removal of heat generated by the one or more light elements.

15. The surgical illumination system of claim 14, wherein the power source is one or more batteries.

16. The surgical illumination system of claim 14, wherein at least one of the one or more illuminating retractor pins includes a heat resistive layer.

17. The surgical illumination system of claim 14, wherein the one or more light elements are light emitting diodes.

18. The surgical illumination system of claim 14, wherein the one or more light elements are organic light emitting diodes.

19. The surgical illumination system of claim 14, wherein the one or more light elements are polymer light emitting diodes.

20. The surgical illumination system of claim 14, wherein the one or more light elements includes at least one array of light elements wherein each light element of the array may be separately energized and each light element of the array produces light of a different frequency.

21. A surgical illumination system comprising:
an elongate surgical pin having a first end and second end;
an illumination collar having a light element mounting surface and frictionally engaging the surgical pin anywhere between the first end and the second end;
one or more light elements mounted in the illumination collar;
an elongate surgical pin having one or more light elements incorporated within the pin;
a power source providing power to each of the one or more light elements; and
a control element controlling the application of power to each of the one or more light elements,
wherein illumination collar position is rotatabily adjustable around the surgical pin, and
wherein the surgical pin acts as a heat sink for optimal removal of heat generated by the one or more light elements.

22. The surgical illumination system of claim 22, wherein the one or more light elements are light emitting diodes.

23. The surgical illumination system of claim 22, wherein the one or more light elements are organic light emitting diodes.

24. The surgical illumination system of claim 22, wherein the one or more light elements are polymer light emitting diodes.

25. The surgical illumination system of claim 22, wherein at least some of the light elements are mounted in a surface which circumscribes the collar.

26. The surgical illumination system of claim 22, wherein at least some of the light elements are mounted in a front surface of the collar.

27. The surgical illumination system of claim 22, wherein at least one of the one or more light elements includes a polarizing film.
